(19) United States
(12) Patent Application Publication
(10) Pub. No.: US 2022/0071569 A1
(43) Pub. Date: Mar. 10, 2022

(54) INFORMATION MANAGEMENT SYSTEM, AND METHOD FOR DEVICE REGISTRATION OF MEASURING DEVICE AND INFORMATION TERMINAL

(71) Applicant: OMRON HEALTHCARE Co., Ltd., Kyoto (JP)

(72) Inventor: Takashi KITAMURA, Kyoto (JP)

(21) Appl. No.: 17/448,138

(22) Filed: Sep. 20, 2021

Related U.S. Application Data
(63) Continuation of application No. PCT/JP2020/009230, filed on Mar. 4, 2020.

(30) Foreign Application Priority Data
Mar. 25, 2019 (JP) 2019-057303

(51) Int. Cl. A61B 5/00; A61B 5/022; H04W 12/50

(52) U.S. Cl. CPC A61B 5/7405; A61B 5/742; H04W 12/50; A61B 5/7475; A61B 5/02233; A61B 5/7455

(57) ABSTRACT

An information management system including a measuring device and an information terminal. In the measuring device, a first communication means transmits identification information identifying the measuring device, and a first output means outputs a unique pattern in a human-perceivable manner at least when the identification information is transmitted. In the information terminal, a second communication means acquires the identification information, a display means displays information related to the measuring device having transmitted the identification information acquired, a second output means outputs, in a human-perceivable manner, a pattern identical to the pattern output by the measuring device displayed, an input means receives an input indicating that the pattern output by the measuring device displayed is identical to the pattern output by the information terminal, and a storage means stores the identification information regarding the measuring device for which the input indicating that the patterns output are identical is provided.

### CROSS-REFERENCE TO RELATED APPLICATIONS

This application is the U.S. national stage application filed pursuant to 35 U.S.C. 365(c) and 120 as a continuation of International Patent Application No. PCT/JP2020/009230, filed Mar. 4, 2020, which application claims priority to Japanese Patent Application No. 2019-057303, filed Mar. 25, 2019, which applications are incorporated herein by reference in their entireties.

### TECHNICAL FIELD

The present invention relates to an information management system including a measuring device and an information terminal, and a method for device registration of the measuring device and the information terminal.

### BACKGROUND ART

In recent years, it has become widespread to perform health management by: measuring personal health-related information (also referred to as “health information” hereinafter), such as weight, blood pressure value, and activity level, by a measuring device; and recording and analyzing the measurement results by an information terminal, such as a smartphone.

When such health management is performed, preferably the measuring device and the information terminal are connected to each other and the result of measurement by the measuring device can be automatically acquired by the information terminal without a user having to input the result to the information terminal each time. Specifically, for example, a method can be considered in which an information terminal receives measurement information from a measuring device by near-field wireless communication, but under circumstances in which a plurality of measuring devices of the same type are used, it is necessary to prevent erroneous reception of information from measuring devices other than the device that has actually performed the measurement.

In order to solve such problems, a predetermined measuring device may be device-registered in a predetermined information terminal, and information may be exclusively acquired from the device-registered measuring device. The device registration is performed by, for example, registering, in an information terminal, identification symbols identifying individual measuring devices, and conventionally a method has been adopted in which identification information printed on a housing of a device or on a seal attached to the housing is manually input to the information terminal.

With such a method, the task is complex for the user and may result in erroneous input. In contrast, Patent Document 1 proposes that serial information provided on a measuring device be acquired by a receiving device by taking a picture or scanning a code and that the serial information acquired be used for device registration.

Additionally, pairing (mutual authentication of devices) has spread that uses wireless communication based on Bluetooth (trade name) (for example, Patent Document 2). In a specific authentication method, pairing is performed such that one of the devices transmits, by advertisement, information indicating that the device is ready for pairing and such that the other device receives the information and designates the first device.

### CITATION LIST

### Patent Literature

Patent Document 1: JP 2016-512965 T

Patent Document 2: JP 2017-170108 A

### SUMMARY OF INVENTION

### Technical Problem

Nevertheless, even in the technology set forth in Patent Document 1 described above, the user must capture an image (or scan) the identification information and thus, in this regard, a complex task arises similarly to the related art. Furthermore, in the technology described in Patent Document 2, in a situation in which a plurality of devices of the same type transmit advertisement information indicating that the devices are ready for pairing, a user fails to determine which of candidates displayed on an information terminal is to be paired and fails to achieve proper pairing.

In light of the related art such as described above, an object of the present invention is to provide a technology that reduces a burden on a user when the user device-registers a measuring device in an information terminal.

### Solution to Problem

In order to achieve the above-described object, an information management system according to the present invention includes one or more measuring devices and one or more information terminals, wherein the measuring device includes: a first communication means for transmitting identification information identifying the measuring device; a first output means for outputting a unique pattern in a human-perceivable manner at least when the identification information is transmitted; and a first control means for controlling the first communication means and the first output means, the information terminal includes a second communication means for receiving the identification information; a second output means for outputting a pattern identical to the unique pattern in a human-perceivable manner, the second output means including a display means for displaying information related to the measuring device having transmitted the identification information; a storage means capable of storing the identification information; an input means for receiving an input from a user; and a second control means for controlling the second communication means, the second output means, the storage means, and the input means, the second control means receives, via the input means, an input indicating that the pattern output by the measuring device displayed on the display means is identical to the pattern output by the second output means, and the storage means is caused to store the identification information regarding the measuring device for which the input indicating that the pattern output by the measuring device is identical to the pattern output by the second output means has been provided.

Note that “unique” described above does not only indicates that a pattern specific to each device is defined in advance but means that at the time of pattern output, the device can be distinguished from the other devices. Thus, the pattern may be a random pattern created each time from an individual seed, an individual random number table, and the like for each device. Additionally, “pattern” as used herein includes a meaning including timing for output, and is not synonymous with frequency. FIG. 11 illustrates an image diagram of a “pattern” of the present specification. The horizontal axis of FIG. 11 represents the time axis. Black squares each indicate the presence of output, and blank portions with no black square indicate the absence of output.

Additionally, the measuring device described above includes various measuring devices such as: body information measuring devices such as a scale, a body composition meter, a blood pressure monitor, a heart rate monitor, and a thermometer; activity measuring devices such as a pedometer and an activity meter provided in various fitness devices; and environmental information measuring devices such as a temperature and humidity meter, a noise meter, and an illuminometer. Additionally, “information terminal” described above may include stationary information terminals in addition to mobile information terminals such as a smartphone, a tablet terminal, and a laptop computer.

In addition, “first communication means” described above may be a transmission means that performs only transmission, or may be a transmission and reception means that can also perform reception. Additionally, “second communication means” described above may be a reception means that performs only reception only, or may be a transmission and reception means that can also perform transmission.

According to the configuration of the system as described above, the human-perceivable, predetermined pattern is simultaneously output by both the measuring device and the information terminal, and thus device registration can be achieved with easy determination of whether the device to be device-registered by the user matches the device indicated in the information terminal as a device registration target.

Additionally, the first communication means may transmit information related to a measurement value measured by the measuring device, and the second communication means may receive the information related to the measurement value measured by the measuring device. Such a configuration allows the information terminal to easily acquire and manage the measurement value measured by the measuring device.

Additionally, the first communication means transmits the identification information by a signal for which transmission and stoppage are repeated in the unique pattern, and the second output means provides, as a pattern, an output indicating whether the second communication means has received the signal transmitted from the first communication means. Thus, the pattern identical to the unique pattern may be output in a human-perceivable manner. In this regard, “providing, as a pattern, an output indicating whether the signal is received” refers to, for example, providing the output when the signal is received, and stopping the output when the signal is not received, thus outputting the pattern based on the time-series presence or absence of the output. Note that the presence or absence of the reception of the signal may oppositely correspond to the presence or absence of the output.

In addition, the first communication means may transmit pattern indication information indicating the unique pattern, the second communication means may receive the pattern indication information, and based on the pattern indication information, the second output means may output a pattern identical to the unique pattern in a human-perceivable manner.

The configuration described above eliminates the need for the information terminal to hold the information of the pattern associated with the identification information, and even in a case where the measuring device generates a unique pattern each time the identification information is transmitted, the information terminal can output the pattern identical to the pattern output by the measuring device.

Additionally, the first output means may include a first sound-wave generating means, and the first sound-wave generating means may output sound of the unique pattern by using a sound wave in an audible range. Such a configuration enables the user to auditorially check the pattern output by the measuring device even without viewing the measuring device, allowing the user to perform a device registration operation with focus placed only on the information terminal. This enables a reduction in complicatedness to the user.

Additionally, the first output means may include a first image display means, and the first image display means may output display that varies in the unique pattern. Such a configuration enables the user to perform the device registration operation by viewing both the measuring device and the information terminal, thus allowing the user to perform device registration even in a situation where generating sound is not appropriate.

Additionally, the first output means may include a first light emitting means, and the first light emitting means may output light that blinks in the unique pattern. Note that the blink as used herein does not only mean a change in light between turn-on and turn-off, but also includes a change in brightness. Such a configuration allows the user to recognize the pattern output by the measuring device without gazing the measuring device as long as blinks of light are in view, allowing the user to perform the operation with a line of sight focused on the information terminal. This enables the user to perform device registration even in a situation where generating sound is not appropriate.

Additionally, the second output means may include a second sound-wave generating means, and the second sound-wave generating means may output sound in a pattern identical to the unique pattern by using a sound wave in an audible range. Such a configuration allows the user to check the pattern output by the information terminal even without a need to view the information terminal.

Additionally, the second output means may include a second image display means, and the second image display means may output display that varies in a pattern identical to the unique pattern. Such a configuration allows the user to check the display means of the information terminal for device registration, while simultaneously checking the output pattern from the information terminal. Additionally, even in a situation where generating sound is not appropriate, device registration can be performed without generation of sound.

Additionally, the second output means may include a second light emitting means, and the second light emitting means may output light that blinks in a pattern identical to the unique pattern. Such a configuration enables the display means of the information terminal for the device registration operation to be separated from the display means for pattern output, thus allowing the pattern to be visually checked without complicating the display contents of the display means for device registration.

Additionally, the second output means may include a vibrator, and the vibrator may vibrate the information terminal in a pattern identical to the unique pattern. Such a configuration allows the user to check the output pattern from the information terminal without viewing the information terminal even in a situation where generating sound is inappropriate.

Additionally, the first communication means may include an oscillation device capable of outputting ultrasonic waves, and the first communication means may transmit the identification information using ultrasonic waves, and the second communication means may include a microphone capable of detecting ultrasonic waves, and the second communication means may receive the identification information by detecting the ultrasonic waves. Such a configuration allows the target measuring device for device registration to be searched for through ultrasonic communication.

Additionally, the first communication means and the second communication means may include a wireless communication means capable of transmitting and receiving information to and from each other, and pairing between the measuring device and the information terminal may be established by causing the storage means to store the identification information.

Additionally, a method for device registration according to the present invention is a method for device-registering a measuring device in an information terminal, the method including the steps of: the measuring device transmitting identification information identifying the measuring device; the measuring device outputting a unique pattern in a human-perceivable manner; the information terminal receiving and acquiring the identification information; the information terminal displaying information related to the measuring device having transmitted the identification information acquired; the information terminal outputting, in a human-perceivable manner, a pattern identical to the pattern output by the measuring device for which the information is displayed on the information terminal; the information terminal receiving an input indicating that the pattern output by the measuring device displayed on the display means is identical to the pattern output by the information terminal; and the information terminal storing the identification information regarding the measuring device for which the input indicating that the pattern output by the measuring device is identical to the pattern output by the information terminal has been provided.

### Advantageous Effects of Invention

According to the present invention, a burden on a user can be reduced when the user device-registers a measuring device in an information terminal.

### BRIEF DESCRIPTION OF DRAWINGS

FIG. 1 is a block diagram illustrating an overview of a configuration example of an information management system according to a first embodiment.

FIG. 2 is a diagram illustrating an example of a display screen of a blood pressure monitor of the information management system according to the first embodiment.

FIG. 3 is a flowchart illustrating a flow of device registration processing in the information management system according to the first embodiment.

FIG. 4A is a first diagram illustrating an example of a change in display of a smartphone in the information management system according to the first embodiment. FIG. 4B is a second diagram illustrating an example of a change in display of the smartphone in the information management system according to the first embodiment.

FIG. 5 is a diagram illustrating an example of a screen display of a smartphone in the information management system according to the first embodiment.

FIG. 6 is a diagram illustrating a configuration example of an information management system according to a second embodiment.

FIG. 7 is a flowchart illustrating a flow of device registration processing in the information management system according to the second embodiment.

FIG. 8 is a diagram illustrating an example of a screen display of the smartphone in the information management system according to the second embodiment.

FIG. 9 is a block diagram illustrating an overview of a configuration example of an information management system according to a third embodiment.

FIG. 10 is a flowchart illustrating a flow of pairing processing in the information management system according to the third embodiment.

FIG. 11 is an explanatory diagram illustrating an image of a pattern of the present specification.

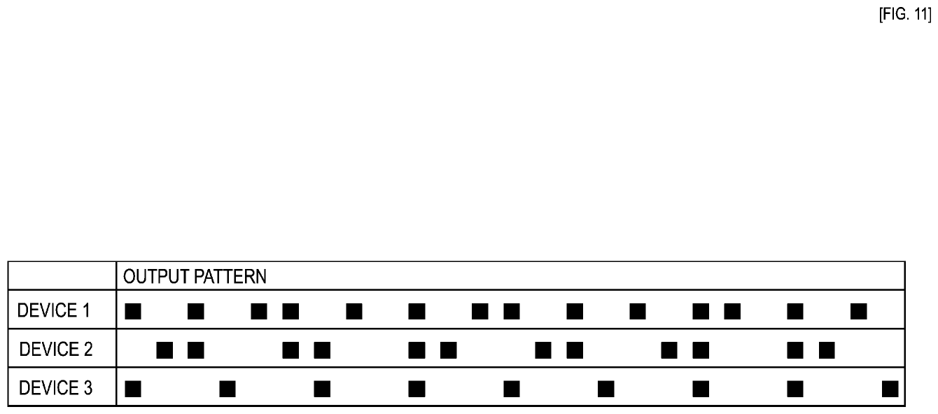

### DESCRIPTION OF EMBODIMENTS

Specific embodiments of the present invention will be described below with reference to the drawings.

### First Embodiment

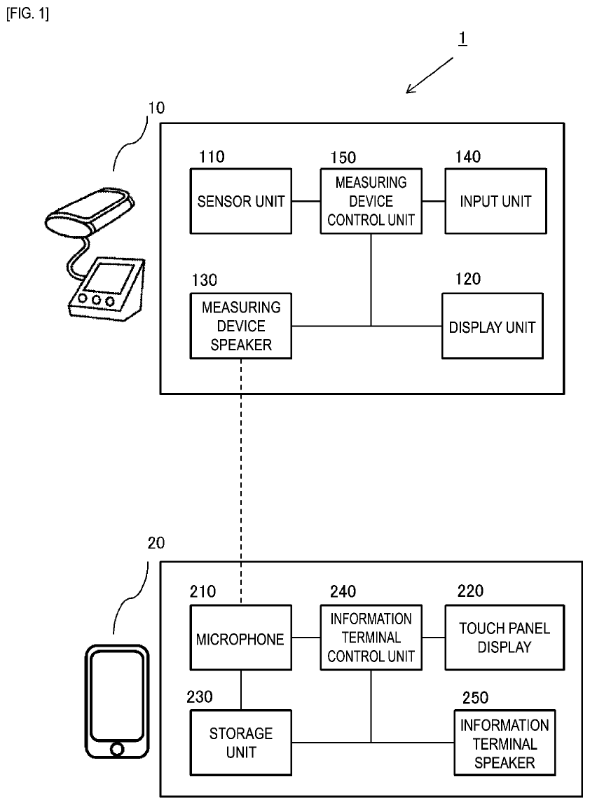

First, an example of the embodiments of the present invention will be described with reference to FIG. 1 to FIG. 5. However, the dimensions, materials, shapes, relative arrangements, and other characteristics of the components described in this embodiment are not intended to limit the scope of this invention unless otherwise indicated.

### System Configuration

FIG. 1 is a schematic diagram illustrating a configuration example of a health information management system 1 according to the present embodiment. As illustrated in FIG. 1, the health information management system 1 includes a blood pressure monitor 10 used as an example of a measuring device, and a smartphone 20 used as an example of an information terminal. By device-registering measuring devices including the blood pressure monitor 10 as described below, the smartphone 20 can subsequently identify and acquire measurement information regarding the device-registered devices.

### Measuring Device

The blood pressure monitor 10 of the present embodiment is a measuring device that measures the blood pressure of a user using a so-called oscillometric method and as illustrated in FIG. 1, includes a sensor unit 110, a display unit 120, a measuring device speaker 130, an input unit 140, and a measuring device control unit 150.

The sensor unit 110 includes a pressure sensor disposed in a cuff portion of the blood pressure monitor 10, and detects a pulse wave from a blood vessel of the user under an appropriate cuff pressure. The blood pressure monitor 10 of the present embodiment is capable of measuring a pulse in addition to a systolic blood pressure and a diastolic blood pressure on the basis of the pulse wave detected by the sensor unit. The values of the systolic blood pressure, the diastolic blood pressure, and the pulse are collectively referred to as measurement values.

The display unit 120 is formed by, for example, a liquid crystal display, and displays the operating condition of the device and the like as well as calculated measurement values. Note that, in the present embodiment, the display unit 120 corresponds to a first image display means.

The measuring device speaker 130 is an output means capable of generating sound waves, and can output ultrasonic waves as well as sound waves in the audible range (hereinafter, sound). As described below, by causing the measuring device speaker 130 to intermittently generate ultrasonic waves in a unique pattern under the control of the measuring device control unit 150, identification information identifying the blood pressure monitor 10 can be transmitted. In other words, in the present embodiment, the measuring device speaker 130 is configured to function as a first communication means and a first sound-wave generating means (first output means) alike.

The input unit 140 is an input means such as a button or a touch panel display that receives input from the user, and receives various operations from the user, such as those for turning the power source on or off, starting measurement, changing a mode, and selecting an item.

The measuring device control unit 150 is a means for controlling the blood pressure monitor 10, and includes a central processing unit (CPU), for example. In response to reception of the execution of the device information registration mode from the user via the input unit 140, the measuring device control unit 150 causes the measuring device speaker 130 to intermittently generate ultrasonic waves in a unique pattern. The ultrasound waves output in the pattern include the identification information regarding the blood pressure monitor 10. Note that the identification information may be held by a storage means or the like (not illustrated), for example.

In addition, the measuring device control unit 150 causes the measuring device speaker 130 to generate sound in the same output pattern as that of the ultrasonic output while the device information registration mode is in execution. Specifically, the signal sound may be generated in an identical pattern.

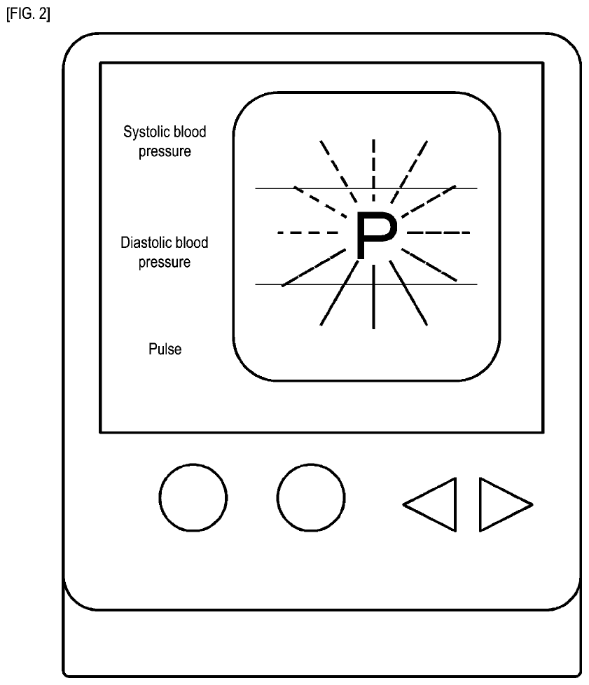

Depending on the selection of the user, as an alternative to or simultaneously with the generation of sound, the display unit 120 may be caused to output display that varies in the same pattern as that described above. For example, a “P” mark may be displayed or disappear in the above-described pattern. FIG. 2 illustrates a display example of the display unit 120 in a pairing mode.

Additionally, a LED light not illustrated may be blinked as an alternative to or simultaneously with the generation of sound or a variation in the display of the display unit 120 as described above. Note that in this case, the LED light corresponds to a first light emitting means.

Note that in the present embodiment, the measuring device control unit 150 corresponds to a first control means, and in addition to performing the above-described control, controls each component of the blood pressure monitor 10 to execute processing in accordance with the operation of the user via the input unit 140.

### Information Terminal

The smartphone 20, used as an example of the information terminal, includes a microphone 210, a touch panel display 220, a storage unit 230, an information terminal control unit 240, and an information terminal speaker 250.

The microphone 210 is one of the input means, and detects sound waves including ultrasonic waves.

As described below, in response to acquisition of the identification information regarding the blood pressure monitor 10 from the ultrasound waves detected by the microphone 210, the touch panel display 220 displays, on the display means of the information terminal, the information regarding the blood pressure monitor 10 having transmitted the identification information. Additionally, display is output that varies in a pattern identical to the pattern of sound waves output by the blood pressure monitor 10 having transmitted the identification information. In addition, operations from the user are received via various input images. In other words, in the present embodiment, the touch panel display 220 is configured to function as a second image display means (second output means) and an input means.

The storage unit 230 includes, for example, a long-term storage medium such as a flash memory, in addition to a main storage device such as a Random Access Memory (RAM), and stores various types of information such as the identification information regarding measuring devices including the blood pressure monitor 10 to be device-registered, application programs, and measurement values.

The information terminal speaker 250 corresponds to a second sound-wave generating means according to the present invention, and outputs various types of sound in addition to outputting sound in the same pattern as that of output provided by the blood pressure monitor 10 in a device registration mode.

The information terminal control unit 240 is a means for controlling the smartphone, and includes, for example, a CPU and the like, and executes various programs stored in the storage unit 230 to deliver functions corresponding to the programs. In the present embodiment, the information terminal control unit 240 corresponds to a second control means.

The information terminal control unit 240 acquires identification information identifying the blood pressure monitor 10 from the ultrasonic waves detected by the microphone 210, for example. Additionally, the information regarding the blood pressure monitor 10 for which the identification information has been acquired is displayed on the touch panel display 220, and display is output to the touch panel display 220, the display varying in the same pattern as that of output provided by the blood pressure monitor 10 for which the identification information has been acquired. Note that, as an alternative to or in addition to a variation in the display, other outputs may be provided, such as causing sound to be output from the information terminal speaker 250 in an identical pattern, blinking the LED light (not illustrated), and activating the vibrator.

### Method for Device-Registering Measuring Device in Information Terminal

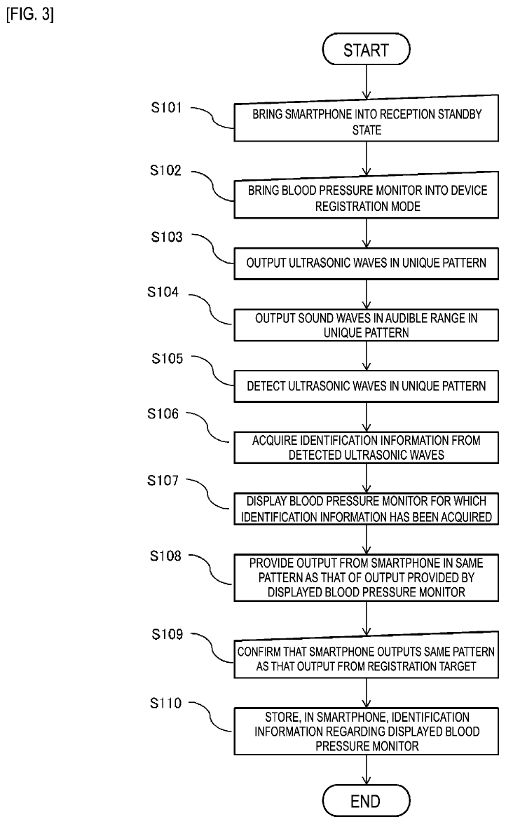

Now, a method for device-registering the blood pressure monitor 10 in the smartphone 20 will be described. FIG. 3 is a flowchart illustrating a processing procedure used when the blood pressure monitor 10 is device-registered in the smartphone 20.

As illustrated in FIG. 3, the user first causes the smartphone 20 to stand by via the touch panel display 220 and the information terminal control unit 240 of the smartphone 20 such that the smartphone 20 is ready for receiving measurement information (step S101). Specifically, for example, an application program for health information management may be executed, and the information terminal control unit 240 may be configured to continually execute the application program in the background.

Then, the user brings the blood pressure monitor 10 into the device registration mode via the display unit 120 and the measuring device control unit 150 of the blood pressure monitor 10 (step S102). In the device registration mode, the measuring device control unit 150 causes the measuring device speaker 130 to output ultrasonic waves in a unique pattern (step S103). Note that, as described above, the ultrasonic wave is output including identification information enabling the blood pressure monitor 10 to be identified.

Furthermore, the measuring device control unit 150 causes the measuring device speaker 130 to generate a signal sound with a beat linked with the pattern (step S104). Note that in this case, the measuring device control unit 150 may cause the display unit to output display that varies in the same pattern as that described above. Furthermore, the LED light (not illustrated) may be caused to blink in accordance with the pattern.

Then, the microphone 210 of the smartphone 20 in an information wait state detects the ultrasonic waves output in step S103 (step S105). Subsequently, the information terminal control unit 240 acquires the identification information regarding the blood pressure monitor 10 included in the ultrasonic waves detected in step S105 (step S106).

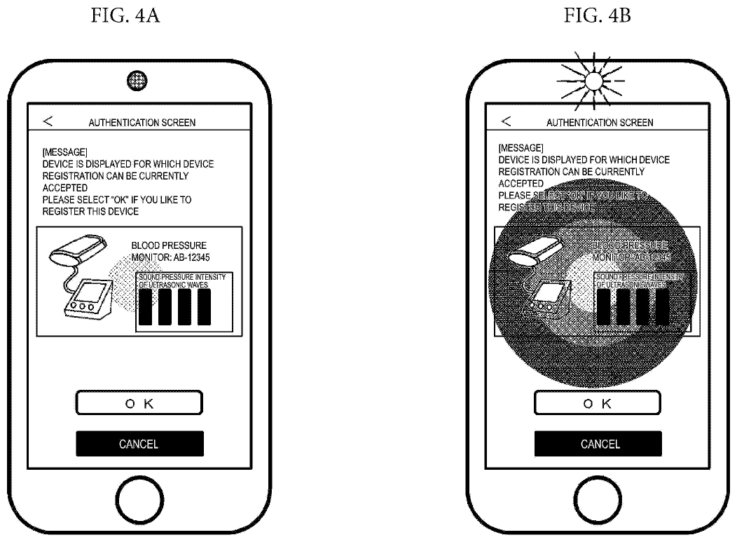

Then, the information terminal control unit 240 displays, on the touch panel display 220, information related to the blood pressure monitor 10 for which the identification information has been acquired (step S107), and outputs, to the touch panel display 220, image display that varies in the same pattern as that of the sound (and other outputs) output from the blood pressure monitor 10 (step S108). Note that, at this time, the information terminal control unit 240 may cause the information terminal speaker 250 to output sound in the same pattern as that described above, or may cause another output means not illustrated to generate an output in the same pattern as that described above. Specifically, for example, the LED light may be caused to blink in the same pattern as that described above, or the vibrator may be caused to vibrate in the same pattern as that described above. FIG. 4 illustrates an example of a case in which a variation in animation exhibiting a pattern is displayed on the touch panel display 220. FIG. 4A is a diagram illustrating an example of a display screen for timing involving no output, and FIG. 4B is a diagram illustrating an example of a display screen for timing involving output, and illustrating the simultaneous turn-on of the LED light in the touch panel display 220.

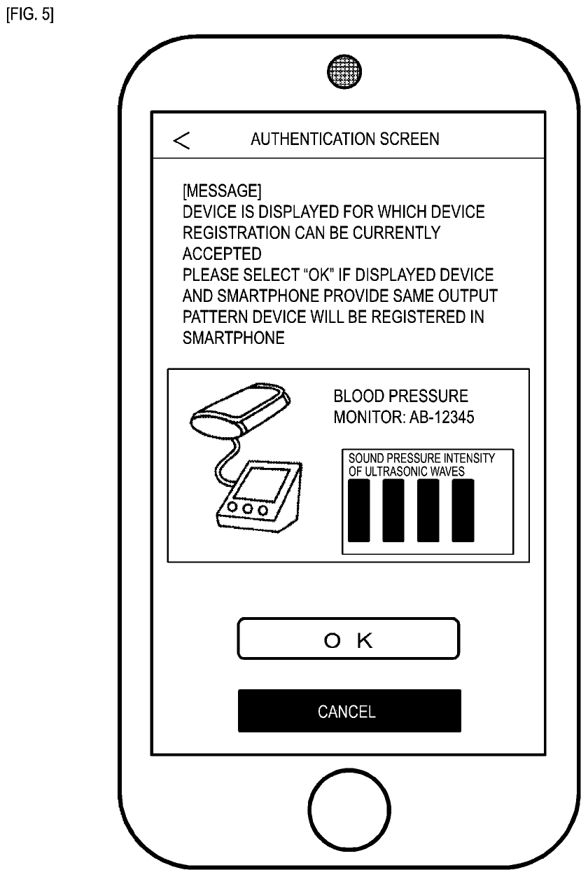

In addition, the information terminal control unit 240 prompts the user to provide an input indicating that the output pattern of the sound output from the blood pressure monitor 10 is identical to the variation pattern of the display of the touch panel display 220 of the smartphone 20 (and other outputs) in a case where the identicalness is confirmed (step S109). Specifically, for example, the touch panel display 220 may be caused to display an operation button for providing an input indicating that the patterns match. FIG. 5 illustrates an example of a display screen for receiving the input of the user in step S109.

A message field of the screen illustrated in FIG. 5 indicates that “A device currently ready for device registration is displayed. In a case where the displayed device and the smartphone provide the same output pattern, please select ‘OK’. The device will be registered in the smartphone.” In other words, in a case where an input is provided indicating that the output pattern from the blood pressure monitor 10 is identical to the output pattern from the smartphone 20, information is provided that indicates that the blood pressure monitor 10 is registered in the smartphone 20.

In step S109, after receiving the input indicating that the output pattern of the sound from the blood pressure monitor 10 is identical to the variation pattern of the display of the touch panel display 220 of the smartphone 20, the information terminal control unit 240 causes the storage unit 230 to store the identification information acquired in step S106 (step S110), and the series of steps of processing is ended.

The storage of the identification information in step S110 identifiably registers the specific blood pressure monitor 10 in the smartphone 20, completing the device registration. After such device registration is performed, the information terminal control unit 240 can exclusively acquire measurement values transmitted from the blood pressure monitor 10 transmitting the identification information stored in the storage unit 230.

Note that in the series of flows described above, the processing in step S103 and the processing in step S104 may be performed in reverse order or concurrently.

According to the configuration of the system as described above, in order to register the measuring device in the information terminal, the user may simply bring the measuring device and the information terminal into the device registration mode and check whether the measuring device and the information terminal output the identical pattern, leading to a very low burden on the user for device registration. In addition, the pattern is output in a human perceivable manner, and thus the user can determine whether the measuring device being device-registered through the information terminal is really the device the user likes to device-register, allowing an uneasy feeling of the user to be removed. Furthermore, the identification information is transmitted by using ultrasonic waves output from the speaker, eliminating the need to separately mount an apparatus for near-field wireless communication. This enables a reduction in the cost of devices constituting the system.

### Second Embodiment

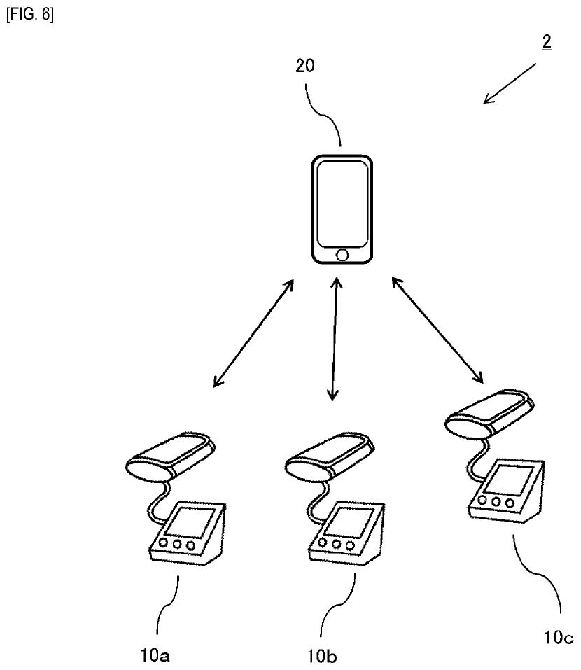

Now, another embodiment of the present invention will be described with reference to FIG. 6 to FIG. 8. In the following, components that are the same as those in the first embodiment are given the same reference numerals, and detailed descriptions thereof will be omitted.

FIG. 6 is a schematic diagram illustrating a configuration example of a health information management system 2 according to the present embodiment. As illustrated in FIG. 6, the health information management system 2 includes a plurality of blood pressure monitors 10a, 10b, and 10c and the smartphone 20. Note that the components of each of the blood pressure monitors 10a, 10b, and 10c, and the smartphone 20 are similar to those in the first embodiment, and therefore descriptions of the components will be omitted.

The health information management system 2 in the present embodiment, unlike in the first embodiment, includes the plurality of blood pressure monitors, and thus the smartphone 20 may receive measurement information from a blood pressure monitor that is different from the blood pressure monitor to be device-registered by the user. Thus, in the processing executed when the blood pressure monitor 10 and the smartphone 20 are paired, a procedure different from that of the first embodiment is executed.

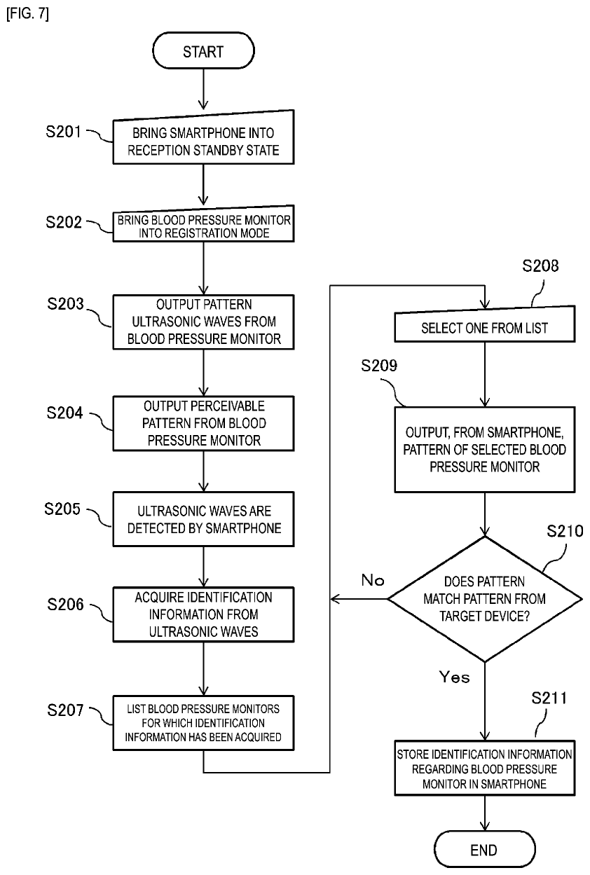

FIG. 7 is a flowchart illustrating a processing procedure executed in the health information management system 2 of the present embodiment when the blood pressure monitors 10 is device-registered in the smartphone 20. The processing from step S201 to step S206 is similar to the processing from step S101 to step S106 in the first embodiment. In other words, the user brings the smartphone 20 into a standby state in step S201 and brings the blood pressure monitor 10 into the device registration mode in step S202. Then, in step S203, the measuring device control unit 150 causes the blood pressure monitor 10 to generate ultrasound waves in a unique pattern, and in step S204, causes the blood pressure monitor 10 to generate a perceivable output with the same pattern as that described above. Furthermore, in step S205, the information terminal control unit 240 causes the microphone 210 to detect ultrasonic waves, and in step S206, acquires the identification information included in the ultrasonic waves detected.

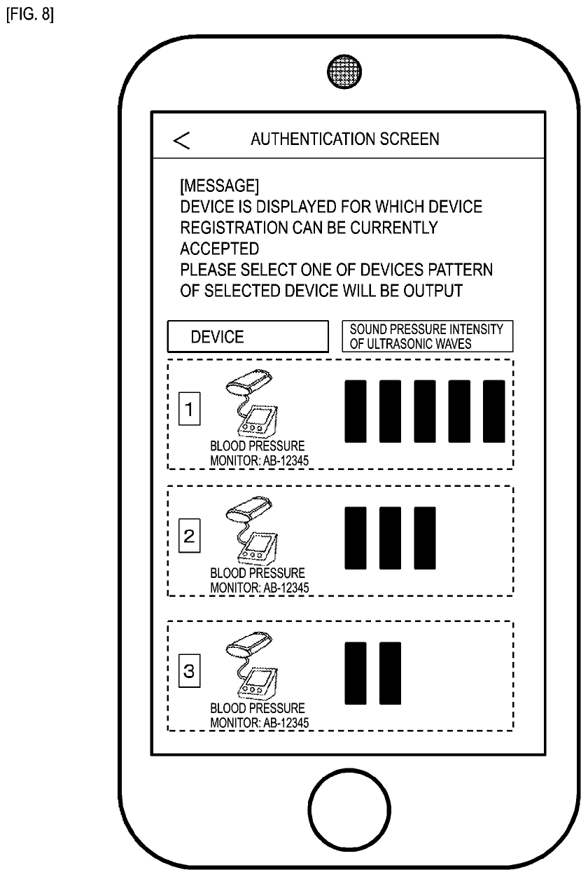

Then, the information terminal control unit 240 causes the touch panel display 220 to display a list of blood pressure monitors 10 having transmitted the identification information acquired in step S206, and prompts the user to select one of the devices (step S207). FIG. 8 is an example of a display screen of the touch panel display 220 displaying a list in response to acquisition of the identification information from a plurality of the blood pressure monitors 10. As illustrated in FIG. 8, in a case where the identification information is acquired from the plurality of measuring devices, the devices may be listed in order of intensity of sound pressure of the ultrasonic waves by which the identification information is transmitted. The intensity of the sound pressure and the distance to the source of the ultrasonic waves are correlated, and thus more intense sound pressure can be estimated to indicate a closer device, and this can be used as a reference for selection of a device.

When the user selects any one device from among the blood pressure monitors 10 listed (step S208), the information terminal control unit 240 displays, on the touch panel display 220, information related to the selected device, and outputs, to the touch panel display 220, image display that varies in the same pattern as that output from the blood pressure monitor 10 (step S209). In this regard, a variation in the display output to the touch panel display 220 may be similar to that illustrated in the first embodiment.

Then, the information terminal control unit 240 prompts the user to provide an input indicating whether the pattern output to the smartphone 20 in step S209 is identical to the output pattern from the blood pressure monitor 10 selected in step S208 (step S210). In this case, the user may be prompted to provide an input by using an example of a display screen similar to that illustrated in FIG. 5, for example. In step S210, in a case where an input indicating that the patterns are not identical is provided, then the processing returns to step S208 and the subsequent processing is repeated. On the other hand, in step S210, in a case where an input indicating that the patterns are identical is provided, then the information terminal control unit 240 causes the storage unit 230 to store the identification information regarding the blood pressure monitor 10 (step S211), and the series of steps of processing is ended.

The storage of the identification information in step S211 identifiably registers the specific blood pressure monitor 10 in the smartphone 20, completing the device registration. After such device registration is performed, the information terminal control unit 240 can exclusively acquire measurement values transmitted from the blood pressure monitor 10 transmitting the identification information stored in the storage unit 230.

According to the configuration as described in the present embodiment, in an information management system in which a plurality of measuring devices of the same type are used, even in a case where any measuring devices are in the device registration mode at the same time, one of the measuring device can be selected based on the perceivable pattern. This allows a wrong measuring device to be prevented from being device-registered.

### MODIFIED EXAMPLE

Note that in the second embodiment described above, in step S207, the blood pressure monitors 10 are listed in order of intensity of sound pressure of the ultrasonic waves detected by the smartphone 20. However, this is not necessary, and for example, the blood pressure monitors may be listed in the order in which the identification information is acquired. In addition, the blood pressure monitors 10 need not necessary be listed, and for example, only the blood pressure monitor 10 for which the detected ultrasound waves have the most intense sound pressure may be displayed as a first candidate, with the same pattern as the output pattern from this blood pressure monitor 10 being output from the smartphone 20. In this case, the display screen as illustrated in FIG. 5 may prompt the user to provide an input indicating whether the patterns are identical, and in a case where an input indicating that the patterns are not identical is provided, for example, “Cancel” is selected, then the blood pressure monitor with the second most intense sound pressure may be displayed as a second candidate.

### Third Embodiment

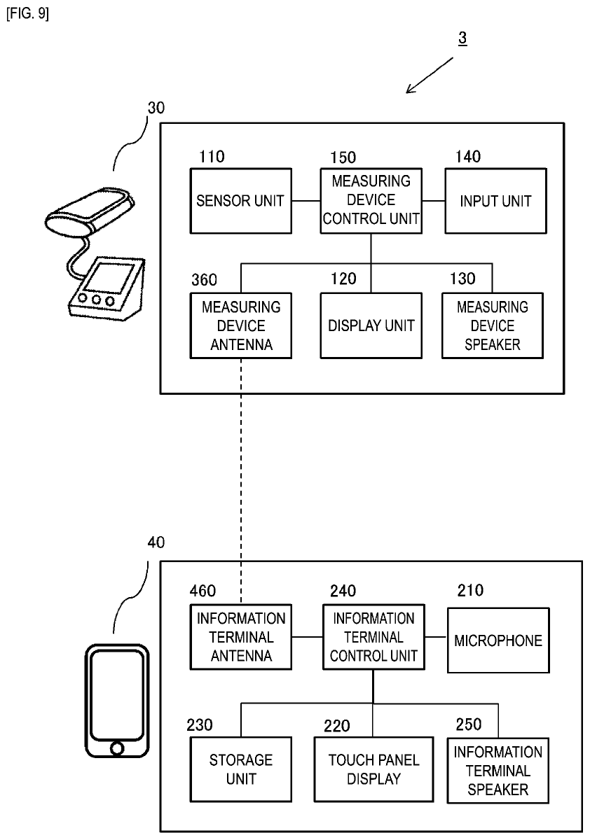

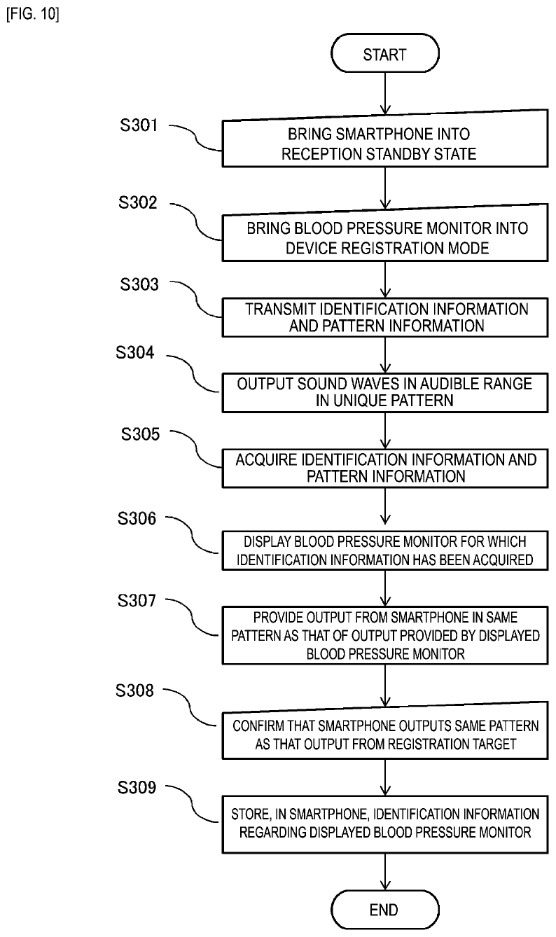

Note that in the above-described first and second embodiments, the identification information is transmitted by using ultrasonic waves but that the identification information can be transmitted without using ultrasonic waves, with device registration performed. Now, another embodiment of the present invention will be described with reference to FIGS. 9 and 10. In the following, components that are the same as those in the first embodiment are given the same reference numerals, and detailed descriptions thereof will be omitted. FIG. 9 is a schematic diagram illustrating a configuration example of a health information management system 3 according to the present embodiment. As illustrated in FIG. 1, the health information management system 3 includes a blood pressure monitor 30 used as an example of the measuring device, and a smartphone 40 used as an example of the information terminal.

The blood pressure monitor 30 differs from the blood pressure monitor 10 according to the first embodiment in that the blood pressure monitor 30 includes a measuring device antenna 360, and is otherwise similar to the blood pressure monitor 10. Additionally, the smartphone 40 also differs from the smartphone 20 of the first embodiment in that the smartphone 40 includes an information terminal antenna 460, and is otherwise similar to the smartphone 20.

Each of the measuring device antenna 360 and the information terminal antenna 460 is a communication antenna for bidirectional wireless communication such as Bluetooth, and the blood pressure monitor 30 and the smartphone 40 are configured to enable wireless communication via the respective antennas. Such a configuration allows the blood pressure monitor 30 and the smartphone 40 to communicate wirelessly with each other in both directions and to be paired with each other.

Specifically, the blood pressure monitor 30 and smartphone 40 in the present embodiment establish pairing after determining the blood pressure monitor 30 to be device-registered in the smartphone 40 using the following method. FIG. 10 is a flowchart illustrating a processing procedure used in the health information management system 3 of the present embodiment when the blood pressure monitor 30 is device-registered in the smartphone 40.

As illustrated in FIG. 10, the user first causes the smartphone 40 to stand by via the touch panel display 220 and the information terminal control unit 240 of the smartphone 40 such that the smartphone 40 is ready for receiving the measurement information (step S301).

Then, the user brings the blood pressure monitor 30 into the device registration mode via the display unit 120 and the measuring device control unit 150 of the blood pressure monitor 30 (step S302). In the device registration mode, the measuring device control unit 150 transmits, via the measuring device antenna 360, identification information enabling the blood pressure monitor 30 to be identified and information indicating a unique pattern associated with the identification information (hereinafter, also referred to as pattern indication information). In other words, in the present embodiment, the measuring device antenna 360 corresponds to the first communication means.

Furthermore, the measuring device control unit 150 causes the measuring device speaker 130 to generate sound in a unique pattern (step S304). Note that in this case, the measuring device control unit 150 may cause the display unit to output display that varies in the same pattern as that described above. Furthermore, the LED light (not illustrated) may be caused to blink in accordance with the pattern.

Then, the identification information regarding the blood pressure monitor 30 is acquired by the information terminal antenna 460 of the smartphone 40 in the information wait state by receiving the identification information and the pattern indication information (step S305). In other words, in the present embodiment, the information terminal antenna 460 corresponds to a second communication means.

Then, the information terminal control unit 240 displays, on the touch panel display 220, information related to the blood pressure monitor 30 for which the identification information has been acquired (step S306), and outputs, to the touch panel display 220, image display that varies in the pattern indicated by the pattern indication information (step S307). Note that because the blood pressure monitor 30 also outputs sound from the measuring device speaker 130 in a pattern identical to the pattern indicated by the pattern indication information, the output of the sound and the variation in the image displayed on the touch panel display 220 indicate the identical pattern.

Furthermore, the information terminal control unit 240 prompts the user to provide an input indicating that the output pattern of the sound output from the blood pressure monitor 30 is identical to the variation pattern of the display of the touch panel display 220 of the smartphone 40 in a case where the identicalness is confirmed (step S308). Specifically, for example, the touch panel display 220 may be caused to display an operation button for providing an input indicating that the patterns match.

In step S308, after receiving the input indicating that the output pattern of the sound from the blood pressure monitor 30 is identical to the variation pattern of the display of the touch panel display 220 of the smartphone 40, the information terminal control unit 240 causes the storage unit 230 to store the identification information acquired in step S305 (step S309), and the series of steps of processing is ended. Note that in the series of flows described above, the processing in step S303 and the processing in step S304 may be performed in reverse order or concurrently.

The storage of the identification information in step S309 establishes the pairing between the blood pressure monitor 30 and the smartphone 40. In this manner, after pairing is established between the blood pressure monitor 30 and the smartphone 40, bidirectional information communication between the blood pressure monitor 30 and the smartphone 40 is enabled by wireless communication via the measuring device antenna 360 and the information terminal antenna 460.

According to the configuration as described in the present embodiment, output of a human-perceivable pattern enables confirmation of a target device to be paired before determination of a pairing partner, allowing the measuring device and the information terminal to be accurately paired.

### Other

The description of each of the examples described above is merely illustrative of the present invention, and the present invention is not limited to the specific embodiments described above. Within the scope of the technical idea of the present invention, various modifications and combinations may be made.

For example, the measuring device may be a body information measuring device other than a blood pressure monitor, such as a scale, a body composition meter, a pulse meter, or a thermometer. Additionally, the measuring device may be an activity meter that measures the quantity of exercise in a pedometer, a tread mill, an Aerobike (trade name), or the like. In this case, the measured quantity displayed on the display unit may be the number of steps, a travel (walking) distance, or the like, may be a value such as the estimated number of calories consumed, or both. Additionally, the measuring device may be an environment sensor device that measures environmental information such as room temperature, humidity, noise, and illuminance. Note that while, in the example described above, the system includes only one type of measuring device that is the blood pressure monitor, the system may be configured to include a plurality of different measuring devices.

Additionally, the information terminal is not limited to a smartphone, and may be another mobile information terminal such as a tablet terminal, or may be a stationary terminal.

In addition, in each of the embodiments described above, the unique pattern is output in conjunction with the transmission of the identification information in the device registration mode. However, the pattern output in the measuring device need not be limited to the above-described pattern output. For example, the output is not limited to the time when the identification information is transmitted, and may be again enabled by the user instruction or the like even after a certain amount of time has passed after the transmission.

Additionally, in the embodiments described above, the output of the sound pattern includes a signal sound. However, the output pattern of the sound is not limited to the signal sound, and may be expressed in a piece of music, a character string (words), and the like. In other words, in a case where a piece of music or the like provided by the measuring device matches a piece of music or the like provided by the information terminal, the measuring device may be registered in the information terminal.

Additionally, for example, in the third embodiment described above, in a case where the information terminal generates an output pattern in response to reception of the pattern indication information, there may be a temporal deviation in output pattern between the measuring device and the smartphone. Thus, the effects of the temporal deviation may be suppressed by methods such as increasing the length of the pattern per period, complicating the pattern (e.g., playing a specified melody using an audible sound, etc.), outputting specified colors to each other using color information. In other words, even in a case where a deviation occurs, a difference from a different pattern (e.g., output by another device) may be clarified to allow a temporal deviation between the identical patterns to be distinguished from the deviation from the different patterns. Alternatively, for example, time information may be included in the pattern indication information to eliminate a temporal deviation between the measuring device side and the information terminal side.

Additionally, the blood pressure monitor 30 and the smartphone 40 according to the third embodiment may be applied to the information management system in the second embodiment. Additionally, the wireless communication in the third embodiment is not limited to Bluetooth communication, and may be performed by other wireless communication such as infrared communication.

### REFERENCE SIGNS LIST

1, 2, 3 Information management system
10, 30 Blood pressure monitor
110 Sensor unit
120 Display unit
130 Measuring device speaker
140 Input unit
150 Measuring device control unit
20, 40 Smartphone
210 Microphone
220 Touch panel display
230 Storage unit
240 Information terminal control unit
250 Information terminal speaker
360 Measuring device antenna
460 Information terminal antenna

1. An information management system comprising one or more measuring devices and one or more information terminals, wherein the measuring device includes:
a first communication transceiver configured to transmit identification information identifying the measuring device;
a first output unit configured to output a unique pattern in a human-perceivable manner at least when the identification information is transmitted, the first output unit including at least one of a speaker, a first light emitter, and a first display; and
measuring device processor configured to control the first communication transceiver and the first output unit,
the information terminal includes:
a second communication transceiver configured to receive the identification information;
a second output unit for outputting a pattern identical to the unique pattern in a human-perceivable manner, the second output unit including a second display for displaying information related to the measuring device having transmitted the identification information;
a storage capable of storing the identification information;
an input unit configured to receive an input from a user, the input unit including at least one of a button and a touch panel; and
an information terminal processor configured to control the second communication transceiver, the second output unit, the storage, and the input unit,
the information terminal processor is configured to receive, via the input unit, an input indicating that the pattern output by the measuring device displayed on the second display is identical to the pattern output by the second output unit, and
the storage is caused to store the identification information regarding the measuring device for which the input indicating that the pattern output by the measuring device is identical to the pattern output by the second output unit has been provided.

2. The information management system according to claim 1, wherein
the first communication transceiver is further configured to transmit information related to a measurement value measured by the measuring device, and
the second communication transceiver is further configured to receive the information related to the measurement value measured by the measuring device.

3. The information management system according to claim 1, wherein
the first communication transceiver is further configured to transmit the identification information by a signal for which transmission and stoppage are repeated in the unique pattern, and
the second output unit provides, as a pattern, an output indicating whether the second communication transceiver has received the signal transmitted from the first communication transceiver, the pattern output in a human-perceivable manner, the pattern identical to the unique pattern.

4. The information management system according to claim 1, wherein
the first communication transceiver is further configured to transmit pattern indication information indicating the unique pattern,
the second communication transceiver is further configured to receive the pattern indication information, and
based on the pattern indication information, the second output unit outputs a pattern identical to the unique pattern in a human-perceivable manner.

5. The information management system according to claim 1, wherein
the first output unit includes a first sound-wave generator, and
the first sound-wave generator is configured to output sound of the unique pattern by using a sound wave in an audible range.

6. The information management system according to claim 1, wherein
the first image display outputs display that varies in the unique pattern.

7. The information management system according to claim 1, wherein
the first light emitter is configured to output light that blinks in the unique pattern.

8. The information management system according to claim 1, wherein
the second output unit includes a second sound-wave generator, and
the second sound-wave generator is configured to output sound in a pattern identical to the unique pattern by using the sound wave in an audible range.

9. The information management system according to claim 1, wherein
the second image display outputs display that varies in a pattern identical to the unique pattern.

10. The information management system according to claim 1, wherein
the second output unit includes a second light emitter, and
the second light emitting emitter is configured to output light that blinks in a pattern identical to the unique pattern.

11. The information management system according to claim 1, wherein
the second output unit includes a vibrator, and
the vibrator vibrates the information terminal in a pattern identical to the unique pattern.

12. The information management system according to claim 1, wherein
the first communication transceiver includes an oscillation device capable of outputting ultrasonic waves, and the first communication transceiver transmits the identification information using ultrasonic waves, and
the microphone of the second communication transceiver is capable of detecting ultrasonic waves, and the second communication transceiver receives the identification information by detecting the ultrasonic waves.

13. The information management system according to claim 1, wherein
the first communication transceiver and the second communication transceiver include a wireless communication means unit capable of transmitting and receiving information to and from each other, and pairing between the measuring device and the information terminal is established by causing the storage to store the identification information.

14. The information management system according to claim 1, wherein
the measuring device is one of a scale, a body composition meter, a blood pressure monitor, a pulse meter, a thermometer, and an activity meter.

15. A method for device-registering a measuring device in an information terminal, the method comprising the steps of:
the measuring device transmitting identification information identifying the measuring device;
the measuring device outputting a unique pattern in a human-perceivable manner;
the information terminal receiving and acquiring the identification information;
the information terminal displaying information related to the measuring device having transmitted the identification information acquired;
the information terminal outputting, in a human-perceivable manner, a pattern identical to the pattern output by the measuring device for which the information is displayed on the information terminal;
the information terminal receiving an input indicating that the pattern output by the measuring device displayed on the information terminal is identical to the pattern output by the information terminal; and
the information terminal storing the identification information regarding the measuring device for which the input indicating that the pattern output by the measuring device is identical to the pattern output by the information terminal has been provided.
